(19) United States
(12) Patent Application Publication
(10) Pub. No.: US 2009/0035382 A1
(43) Pub. Date: Feb. 5, 2009

(54) METHOD AND COMPOUND FOR THE TREATMENT OF ARTICULAR DISEASES OR ARTICULAR PAIN, OR FOR THE TREATMENT OF SKIN FOR AESTHETIC OR OTHER PURPOSES, AND THE METHOD OF PREPARATION OF THE COMPOUND

(75) Inventors: Eduardo ANITUA ALDECOA, Vitoria (ES); Mikel Sanchez Alvarez, Vitoria (ES)

Correspondence Address: BROWDY AND NEIMARK, P.L.L.C.;624 NINTH STREET, NW, SUITE 300, WASHINGTON, DC, 20001-5303, US

(73) Assignee: Biotechnology Institute, I MASD, S.L., Vitoria (Alava) (ES)

(21) Appl. No.: 12/185,714

(22) Filed: Aug. 4, 2008

(30) Foreign Application Priority Data
Aug. 2, 2007 (ES) P200702174

(51) Int. Cl. A61K 35/16; A61P 19/02

(52) U.S. Cl. 424/530

(57) ABSTRACT

Method for the treatment of articular diseases or articular pain, or for the treatment of skin, which comprises the infiltration or the application of a compound that comprises at least one blood-derived substance, which is preferably a blood plasma in general, and in particular a platelet-rich plasma (PRP), and preferably a plasma rich in growth factors (PRGF), and/or a supernatant of any of the aforementioned plasmas. The inventive method enables the significant relief and even regeneration of the treated areas. Optionally, the blood-derived substance may be mixed with hyaluronic acid (HA) and/or an HA-derived compound, thereby achieving even more beneficial effects.

### TECHNICAL FIELD

The invention relates to a method for treating articular diseases or articular pain (particularly, for the treatment of osteoarthritis) and to a method for the treatment of skin for aesthetic or other purposes. The invention also relates to a compound that may be used in both methods, and to the method of preparation of said compound.

### PRIOR ART

In recent years, significant advances have been made in the field of regenerative medicine as a result of the search for new treatments that reduce or prevent the deterioration of tissue. Tissue can deteriorate due to wear, ageing or disease. A very important part of these advances involves the development of certain preparations or compounds known as “autologous” compounds. Autologous compounds are characterized in that they derive from the patient him/herself (e.g. from the blood of the patient). Autologous compounds have been increasingly developed in search for medical solutions that use the patient's own resources, in order to avoid the chance of rejection. Additionally, it has been observed in clinical practice that the use of autologous compounds instead of compounds made from generic substances (those not obtained from the patient) also improves and accelerates tissue repair, due to the fact that they provide bioactive molecules that participate in and speed up natural repair processes. Autologous compounds are characterised in that they are biocompatible; in other words, the body recognises them as its own. In addition, autologous compounds are biodegradable, and during their in vivo degradation they release natural subproducts or metabolites that enhance, rather than interfere, with the repair mechanisms (which is not the case with synthetic compounds, which generate products not generated by the body itself when they degrade, potentially giving rise to inflammatory reactions that hamper or even prevent regeneration processes). Autologous compounds are also biomimetic; in other words, their interaction with the host tissues mimics physiological repair mechanisms. Another unusual characteristic of autologous products is their molecular complexity: they are multimolecular compounds, the molecules of which impact on various stages of the repair process. Achieving this effect with synthetic products, in other words, designing and implementing the synthesis of a molecular mixture of these characteristics, proving its efficiency and biosafety by complying with health legislation, and transferring this synthesis to the clinical environment would be a very costly process (in fact, the available conventional treatments are based on a single substance).

Among the autologous compounds used in regenerative medicine, Platelet-Rich-Plasma (PRP) compounds are known. PRP compounds are obtained from the blood of the patient and are used in regenerative medicine mainly because of a particular quality they possess: the localised release (from inside the platelets) of reparative molecules, also known as tissular repair factors. Among the reparative molecules, the very well known cellular signals named generically as “growth factors” are known. Growth factors, which include PDGF, TGF-beta, IGF-I, HGF, VEGF, EGF and bFGF, co-ordinately act on the cells of damaged tissue through specific receptors situated in the cellular membrane. The interaction between the growth factors and the cells is crucial in determining cellular activity. These growth factors may or may not trigger mitosis, modify autogenesis, modify the synthesis of extracellular matrix and the remodelling of the tissue, all of which are processes involved in the regeneration of damaged tissue.

Generally speaking, PRP compounds are obtained from a method of preparation that involves four fundamental steps: the extraction of the patient's blood; the separation of the blood into different parts; the isolation of a platelet-rich part (PRP) from the different parts; and the coagulation (also known as activation) of said PRP so that reparative molecules are released. As a result, a PRP compound with a specific consistency (depending on the degree of coagulation) and biological activity is obtained. These steps known in the prior art can be configured and performed in many different ways, therefore leading to significantly different qualitative and quantitative compositions of PRP compounds. For example, certain known PRP compounds are platelet- and leukocyte-rich plasmas, whereas other known PRP compounds are platelet-rich leukocyte-free plasmas. The absence of leukocytes enables a series of advantageous effects to be achieved. First and foremost, reproducibility of the preparation of these compounds is increased, and the PRP compounds are provided with different and favourable biological characteristics. For example, one of said favourable biological characteristics is the absence of certain leukocytes known as “neutrophils”, which represent 60-70% of all leukocytes and which are considered harmful to tissue under repair as they are a source of free radicals (which indiscriminately destroy local cells) and a source of a type of enzymes known as “metalloproteinases” (specifically MMP-8 and MMP-9, which destroy the components of the extracellular matrix).

PRGF compounds are a type of PRP compound used in regenerative medicine. PRGF compounds are PRP compounds containing a moderate number of platelets, not containing leukocytes, and whose activation, which results in the release of the reparative molecules, is triggered by the addition of calcium chloride. The addition of calcium chloride leads to the formation of a natural polymer known as “fibrin”. As well as providing the aforementioned effects, the absence of leukocytes increases fibrin stability; in other words, it delays fibrin degradation. This is due to the fact that fibrin is free from the effect of the elastases, which are hydrolytic enzymes that destroy the fibrin matrix (accelerating the degradation of said matrix) and which are generated as a consequence of the activation of a leukocyte known as granulocyte.

The state of the art comprises different variations of PRGF compounds, their manufacturing procedures, and their application in various medical situations and in ex vivo cell culture technology. Mainly, the patents U.S. Pat. No. 6,569,204, ES2180438B2 and ES2221770B2 are known. These patents describe procedures for the preparation of a PRGF compound and its use in the regeneration of bone tissue during the insertion of a dental implant or other type of prostheses, as well as its use as collyrium eye drops, and even its use in expanding cells ex vivo. The use of PRGF compounds in these applications, now conventional, has proved to be enormously beneficial.

The invention aims to broaden the field of application and the methods for the use of compounds rich in growth factors (PRGF compounds) in particular, and of platelet-rich compounds (PRP) and blood-derived compounds in general, to applications other than the conventional applications mentioned above.

### BRIEF DESCRIPTION OF THE INVENTION

It is an object of this invention to provide a method for the treatment of articular diseases or articular pain, which comprises the infiltration in the joint of a compound that comprises at least one blood-derived substance. A blood-derived substance possesses biological properties capable of modifying both the biological condition and the mechanical condition of the joint. Said blood-derived substance is preferably a blood plasma and/or a supernatant of blood plasma. The blood plasma is preferably a platelet-rich plasma (PRP), and among PRPs, preferably a plasma rich in growth factors (PRGF).

The method according to the invention enables the treatment of conditions ranging from mild articular pains to diseases such as chondropathia or osteoarthritis.

It is another object of this invention to provide a method for the treatment of skin, for aesthetic or other purposes, which comprises the external application or the infiltration beneath the skin of a compound that comprises at least one blood-derived substance. As in the previous case, said blood-derived substance is preferably a blood plasma and/or a supernatant of blood plasma. The blood plasma is preferably a platelet-rich plasma (PRP), and among PRPs, preferably a plasma rich in growth factors (PRGF).

It is another object of this invention to provide a compound that comprises at least one blood-derived substance, and hyaluronic acid (HA) and/or at least one HA-derived compound. Said compound presents properties that are especially advantageous for the aforementioned applications as it combines the properties of the blood-derived substance itself with the properties of the HA. In addition, it achieves new effects as a result of the combination of both components.

Finally, it is another object of this invention to provide the method of preparation of the previous compound.

### DETAILED DESCRIPTION OF THE INVENTION

It is an object of this invention to provide a method for the treatment of a joint affected by pain or disease, which comprises the infiltration in the joint of a compound that comprises at least one blood-derived substance. According to this inventive method, conditions ranging from mild pains to diseases such as chondropathia or osteoarthritis may be treated.

Osteoarthritis, as is known, is an articular disease with a high incidence and prevalence in the population. 10% of the population over the age of 50 suffer from it. It has no cure, has a major impact on quality of life, is degenerative and also worsens over time. Its social and economic impact is very significant both in terms of lost working hours and health costs. At the present time, palliative treatments are used, aimed at treating the symptoms (mainly the pain). For this purpose, analgesics and anti-inflammatories are administered. However, these treatments neither stop nor alter the course of the disease, which evolves until the joint is seriously deteriorated. The response in such extreme cases is joint replacement through major surgery. Alternatively, treatments that involve the individual application of growth factors such as IGF-I or bFGF (U.S. Pat. No. 6,645,945, US2004017695) are also known. However, they have not yet been used in a clinical environment due to a number of concurrent problems: a lengthy and costly process is required to implement their synthesis and purification in the laboratory, to prove their biosafety and efficiency in vivo, and to comply with the legal health requirements pertaining to synthetic medicines. As regards proving their efficiency, the heterogeneity of patients and the complexity of articular diseases are such that the administering of a single factor intervening in a single mechanism may be insufficient for the modification of a complex pathological process that involves multiple processes.

By means of the treatment according to the invention, which uses a substance with biological properties that is capable of releasing reparative molecules that act in the articular cartilage, new and very significant therapeutic effects are expected to be achieved. These effects include a certain capacity to restore the joint's physiological conditions.

According to the invention, the blood-derived substance may be infiltrated at any moment, in order to relieve articular complaints or injuries of varying degrees of seriousness, or even in other situations. For example, a blood-derived substance may be infiltrated in a joint following an extraction of pathological synovial fluid from the joint, if effusion, inflammation and articular pain occur. The pathological synovial fluid is therefore replaced by a substance that provides a healthier biological intra-articular environment. The blood-derived substance acts on the synovial membrane cells, known as synoviocytes (which are responsible for the production of the synovial fluid that bathes the joint), thereby stimulating the production of hyaluronic acid and other molecules, acting as an anti-inflammatory and reducing the pain. A blood-derived substance may also be infiltrated after arthroscopic surgery on the joint, in order to promote the metabolic activity of the chondrocytes, to inhibit the enzymes that degrade the joint, and to stimulate the production of hyaluronic acid. The blood-derived substance restores the molecular profile and the biological articular environment, which is particularly noteworthy due to the fact that the joint has lost its biological factors as a result of having been washed during arthroscopy.

It is another object of this invention to provide a method for the treatment of skin, which comprises the external application or the infiltration by means of dermal, subdermal or intramuscular injection, of a compound that comprises at least one blood-derived substance.

The blood-derived substance may be autologous (obtained from the patient upon whom the treatment with the end compound is to be applied) or homologous (not obtained from the patient, but from a specimen of its same species). The advantage of the blood-derived substance being autologous is that it can be prepared right when it is to be used (which means that no storage system is required), thereby reducing the cost of the treatment. Furthermore, it is completely biocompatible, i.e. it does not cause any immunological reactions. In contrast, the use of homologous substances may be of interest in the treatment of patients with complex systemic diseases that may affect concentrations of GFs in their blood (generic) and in any of the blood-derived preparations, and who therefore will not benefit from the advantages of this type of treatments. In these cases the situation may be addressed by using a substance derived from the blood of a healthy person with proven reparative properties.

Preferably, the blood-derived compound is a plasma in general, of whatever type, produced in whichever way is required to create a suitable composition for the specific application or clinical case. In other words, a plasma with a specific content of platelets, leukocytes and other components will be used, its composition depending on the method by means of which it is obtained. An advantage of using plasma is that the reparative molecules (growth factors, etc) are released in an order and at a speed that is determined by the natural need of the tissue and the treatment area. In other words, the release of the regenerative substances takes place in a controlled manner (in contrast to other embodiments of the invention, as will be seen at a later stage). As a result, the regeneration process is slower but more complete.

Preferably the blood-derived compound is a platelet-rich plasma (PRP). Platelets are fundamental in repairing tissue as they contain different structures or reparative molecules with bioactive substances that play a crucial role in the repair process. Among these reparative molecules, the aforementioned “growth factors” (PDGF, TGF-beta, IGF-I, HGF, VEGF, EGF, bFGF, etc.) can be found. Growth factors are capable of triggering or not triggering mitosis, of modifying autogenesis, of modifying the synthesis of extracellular matrix and of modifying the remodelling of the tissue.

Particularly advantageous is the embodiment in which, among all known platelet-rich plasmas (PRPs), the invention uses plasma rich in growth factors (PRGF), as this type of PRP compound presents a series of advantages over other PRP compounds that have already been detailed in the introduction to this document. This particular PRGF-based embodiment also presents other advantages related to the articular application. In the first place, this embodiment will contain molecules that block or neutralise the destructive activity of enzymes known as metalloproteinases (MMPs), which are responsible for the destruction of the articular cartilage. For example, PRGF contains TIMPs and alpha-macroglobulin; its application may, therefore, stop the destruction of the cartilage. Secondly, PRGF contains factors such as IGF-I and TGF-beta1, which are anabolic (in other words, they stimulate the synthesis of the matrix) for the cartilage. Treatment with PRGF, therefore, induces the synthesis of new extracellular matrix.

The plasma rich in growth factors (PRGF) may be prepared according to the method disclosed in U.S. Pat. No. 6,569,204 or according to other appropriate methods.

Alternatively or complementarily, the blood-derived substance used in the present invention may consist of or comprise a supernatant originating from the coagulation of a blood plasma. In such a case, unlike the embodiment in which blood plasma is used, the reparative molecules (growth factors, etc.) are already released and ready to act, as a result of which the effect of the supernatant on the tissue or treatment area is quicker. At the same time, the supernatant is fluid, as a result of which it is more dispersed and difficult to confine in the treatment area once it is applied, in contrast with the embodiment in which blood plasma is used.

Particularly advantageously, the blood supernatant is a supernatant obtained from the coagulation of a platelet-rich plasma (PRP). This supernatant presents the advantages associated with the aforementioned properties of the PRP compounds.

Preferably, the supernatant is obtained from the coagulation of a plasma rich in growth factors (PRGF). In this last case, the supernatant may be obtained by means of the procedure disclosed in ES2221770 patent, for example. This supernatant presents the advantages associated with the aforementioned properties of the PRGF compounds.

The invention also contemplates a combination of plasma and supernatant, of whatever type. Because plasmas have a relatively viscous consistency and supernatants have a liquid consistency, the amounts of one another allow to achieve a compound with the specific consistency that is required by the application in which the compound is to be used.

Another object of the invention is a compound that comprises at least one blood-derived substance, be it autologous or homologous, and hyaluronic acid (HA) and/or at least one HA-derived compound. Hyaluronic acid (HA) is a substance used in orthopaedics, traumatology, aesthetic medicine, otolaryngology and opthalmology, among other areas of medicine. The most important feature of HA is its hygroscopic character, namely its attraction to water, as a result of which the HA molecules are capable of immobilising water molecules inside a tissue. Said hygroscopic character is responsible for the volume of the intercellular substance and for the hydration of all tissues. During its degradation, HA does not undergo any variations in volume (it is isovolemic). Due to these qualities, HA is used for fundamentally mechanical purposes as it may provide a filling, control moisture and increase tissue space. It may also be used as protection for delicate areas during surgery, and may replace or supplement biological fluids and provide lubrication (as in documents US20050182022 or U.S. Pat. No. 6,645,945). However, HA has no known biological activity. In other words, it does not interact biologically with the tissue upon which it is applied. Combining, according to the invention, the mechanical, structural and lubricating properties of hyaluronic acid (HA) with the biological properties of blood-derived substances is, therefore, particularly advantageous. As regards its obtainment, HA may be purified from certain animal or human tissue (it is particularly abundant in skin and synovial fluid), or may be obtained by a process of bacterial fermentation and subsequent purification in the laboratory. With regard to its chemical nature, it is a “universal” polysaccharide, as it is identical in all animal species and occupies the intercellular space in all tissue; it is a polymer formed by the repetition of N-acetyl glucosamine and glucuronic acid.

As stated above, according to the invention pure HA or an HA-derived compound may be used. The HA-derived compound may present a number of variations, depending on a series of factors. One factor is the molecular weight of the hyaluronic acid (HA). Another factor is the source of the hyaluronic acid (HA), which may be animal (cock's crest, shark fin, etc.) or another biological source (umbilical cord, eyes, etc.), or which may be a bacterial fermentation, recombinant technologies, etc. An additional factor is whether the hyaluronic acid is modified chemically or chemically-physically by creating crossed links. A further factor is the range of HA-derived compounds it may contain: salts (sodium, potassium, calcium, magnesium, ammonium etc.), esters, hemiesters, partial esters (alginate derivatives, heavy metal salts), sulfated derivatives or N-sulfates, amide derivatives, or with any residue or OH⁻, O⁻ or other substitution group. Finally, another factor is the geometrical shape of the HA, which may be granulated, spherical, powdery, fibrous, spongy, etc.

The compound according to the invention may be used in an especially advantageous form in the treatment of articular diseases or articular pain and in the treatment of skin, for a variety of reasons.
Naturally, use is made of the properties of each component: the natural biological and regenerative qualities of the blood-derived substance, which have been explained above; the qualities of the HA itself (elasticity, viscosity, contribution to lubrication, shock absorption). Furthermore, the fact of combining HA with a blood-derived substance has additional advantageous effects. For example, a more stable compound (less degradation) is obtained than with the blood-derived substance on its own.
A longer temporary presence of HA in the joint is achieved, as an endogenous HA generated by the tissue cells as a consequence of the action of the blood derivative is added to the initial exogenous HA (provided by the compound), which tends to disappear relatively quickly because of its characteristics. In other words, a kind of feedback loop is created: the HA encourages the action of the blood derivative, with the latter ensuring that the former does not disappear.
A greater spatial control of the release and action of the growth factors is achieved, i.e. greater confinement of the growth factors in the treatment area is achieved (in comparison with applying HA-free blood-derived substance). This is due to the isovolemic and hygroscopic nature of the HA, by which it can retain hydrosoluble reparative molecules of the blood-derived substance in said area. In this way, the HA enables the biological activity of the blood-derived substance to be confined long enough in the area where the “target” tissues to be acted upon are located. This effect is advantageous and original in relation to the application of HA-free blood-derived substances. For example, in the event that HA-free PRGF is applied, the network of fibrin contained in the PRGF keeps the reparative molecules confined for a very short period of time: the fibrin retracts and releases the supernatant fluid containing the reparative molecules, it may being the case that said supernatant fluid expands, spreads or leaks towards other areas. In such an outcome, control of the target area would be lost and the reparative molecules would ultimately exert their effect in other areas where they are not required. Logically, this dispersion of the reparative molecules of the HA-free PRGF will occur to a smaller or larger degree depending on the characteristics of the treatment area, the consistency of the PRGF and other factors.
A greater temporary control of the release and action of the growth factors, i.e. a slower release of the reparative molecules in the treatment area, is achieved (in comparison with the application of an HA-free blood-derived substance). As a result, the bioavailability of the reparative molecules is altered. The period of time said reparative molecules remain in the joint is made longer, and the period of time for which the tissue is exposed to the treatment is maximised.

The compound according to the invention provides additional advantages in the treatment of articular diseases in comparison with the exclusive application of a blood-derived substance, for example PRGF. Firstly, the HA and/or HA-derived compound provides a lubricating effect that is very important for a worn joint. Additionally, the inventive compound meets the requirements of an ideal chondroprotective agent, and restores the homeostasis of the joint. Furthermore, by means of the inclusion of HA, the time for which the compound is in contact with the cartilage is increased, as a consequence of which the probability of the penetration and neutralisation of metalloproteinases is greater. Also, as has been stated, the PRGF presents a series of characteristics that make its intraarticular application of interest, the purpose being to prevent the destruction of the cartilage. However, a PRGF compound applied on the cartilage does not meet the requirements of an ideal chondroprotective agent restoring the homeostasis of the joint. PRGF dissociates in a growth-factor-containing supernatant fluid at a faster rate than the rate of penetration of the molecules in the hard matrix of the articular cartilage. For this reason, it is of interest to increase the contact time, as is achieved by means of the HA.

The compound according to the invention also provides specific advantages when applied on the skin (by infiltration or by external application), both in aesthetic medicine and in other fields. Said advantages are based on the fact that the compound performs various functions simultaneously. First of all, it acts as a wrinkle-filling material (by means of the HA). Secondly, the compound regulates hydration, acting as a lubricant (depending on the degree of fluid it contains). Thirdly, the blood-derived substance stimulates the local synthesis of HA and collagen by the dermal fibroblasts, thus resulting in a more stable rejuvenating effect over time. Fourthly, the compound is biocompatible and non-immunogenic (as it is blood derived). Fifthly, the compound is biodegradable. Finally, the viscosity of the compound may vary according to its exact composition and thus be adapted to the different diameters of the injection needles suitable for each application. For example, an application in aesthetic medicine using the technique known as mesotherapy requires many punctures that must not be aggressive at all in nature. Therefore, the viscosity of the compound must be such that it enables the compound to pass through a very thin needle (30 G). In such a case the viscosity must be within a range principally between 2000 ml/g and 20000 ml/g.

The application on the skin of the compound comprising HA and a blood-derived substance may provide better results than the application of only a blood-derived substance, also claimed by the present invention. For example, let it be supposed that the treatment is applied to facial aging. If repeated doses of a PRGF are infiltrated through mesotherapy, the PRGF will act as filling for a very small period of time (a few days), but in return it will initiate cellular processes such as the synthesis of HA and collagen, which result in more stable changes that provide the skin with smoothness, stability and hydration in the long term. In other words, the PRGF would act as a soufflé, its volume disappearing within a short period of time, as a result of which the improved appearance of the patient would not last long. In contrast, the mixture of PRGF+HA will provide a filling effect provided by the HA, and at the same time will trigger cellular activity and set off more stable changes thanks to the bioactivity of the PRGF.

The blood-derived substance used in the inventive compound may be a blood plasma, this blood plasma preferably being a platelet-rich plasma (PRP), and within this plasma type, a plasma rich in growth factors (PRGF). The advantageous properties of each one have already been explained. It is also possible that the blood-derived substance may contain a supernatant of blood plasma, in particular of a platelet-rich plasma (PRP), and preferably of a plasma rich in growth factors (PRGF). Dissolving the compound of hyaluronic acid (HA) in a supernatant leads to a compound that, besides having biological properties (due to the fact that the supernatant originates from a plasma with biological properties), has a liquid or quasi-liquid consistency due to the fact that the supernatant is a liquid compound.

The final consistency of the compound according to the invention may, depending on the proportions of blood-derived substance and HA and/or HA-derived compound it comprises, be liquid (in the event that a large amount of a fluid compound is used qualitatively, e.g. HA in a saline solution and/or supernatant), be a gel (i.e. moderately viscous, by reducing the amount of liquid components), be a membrane (i.e. more viscous, capable of forming a membrane or film), and thereby successively until solid. The chosen final consistency depends on the compound application. For example, in the event that the compound will be used in the treatment of articular diseases, it may present a liquid-viscous consistency so that it can be injected and at the same time coagulate immediately after entering the joint. Alternatively, in the event that the compound is used in a skin treatment, and if the application is external, a liquid or gel consistency is preferable.

An example of an inventive compound is created using a supernatant fluid that contains, among others, PDGF (27.96 ng/ml (SD: 12.13), TGF-b1 (55.27 ng/ml (16.23)), IGF-I (88.62 ng/ml (SD: 38.36)), EGF (456 pg/ml (SD: 210)), VEGF (421 pg/ml (SD: 399)), TIMP-1 (400 ng/ml (SD: 230)), HGF (606 pg/ml (SD: 269)) and which forms a colloid with the HA. Said colloid is mixed with a certain amount of plasma rich in growth factors (PRGF), in a proportion and volume to be referred to as “S/PRGF”. The compound can be configured in a variety of ways, as can be seen in the table below, where the composition of the new multi-molecular compound provided with a multiple-action mechanism, directed at various cellular targets, is described:

| Dispersing phase: | Dispersed phase: HA | PRGF | New compound |
|---|---|---|---|
| supernatant | % weight/vol | % vol/vol | (*) |
| PDGF; | 0.5%-50% | 0.1%-99% | Viscous liquid |
| TGF-b1; | | | Gel |
| IGF-I; | | | Solid |
| EGF; | | | |
| VEGF; | | | |
| TIMP-1; | | | |
| HGF | | | |

(*) Consistency of the new compound according to the quantities of HA and PRGF.

It is another object of this invention to provide a method of preparation of the described compound. Said method of preparation comprises the following steps:
Preparing or beginning with at least one blood-derived substance, autologous or homologous.
Mixing said blood-derived substance with HA and/or at least one HA-derived compound, generally by adding the blood-derived substance by means of a syringe, pipette or other instrument to the HA and/or at least one HA-derived compound, in laminar flow conditions or other similar conditions that guarantee the sterility of the preparation.
Optionally, activating said mixture by means of calcium chloride.

The blood-derived substance is preferably a blood plasma, and especially advantageously a platelet-rich plasma (PRP) and, more particularly, a plasma rich in growth factors (PRGF). The blood-derived substance may also be a supernatant of blood plasma, preferably of a platelet-rich plasma (PRP), and more particularly of a plasma rich in growth factors (PRGF). To prepare any of these supernatants, any conventional method of the types explained in the introduction to this document, or others, may be implemented.

In the event that the blood-derived substance is a non-activated substance (e.g. a blood plasma, a PRP or a PRGF) calcium chloride may be added to activate the substance, in other words to bring about the release of the reparative molecules. However, if the blood-derived substance is an already activated substance (e.g. any of the aforementioned supernatants), it is not essential that calcium chloride be added.

In an embodiment of the method of preparation of the compound the HA and/or HA-derived compound is lyophilised. The mixing of the blood-derived substance with the HA and/or at least one HA-derived compound is carried out by adding the blood-derived substance to a tube containing the lyophilised HA and/or at least one HA-derived compound and heating said tube to between 25 and 42° C. in order to dissolve the HA and/or at least one HA-derived compound in the blood-derived substance.

In another embodiment of the method of preparation of the compound the HA and/or at least one HA-derived compound are initially available in a solution. The mixing of the blood-derived substance with the HA and/or at least one HA-derived compound is carried out by dissolving the HA and/or at least one HA-derived compound in a sterile saline solution at between 25 and 42° C., and then mixing the solution gently with the PRP.

The calcium chloride is preferably applied in a concentration of between 6-120 mM. This range guarantees the activation of the platelets, the formation of fibrin and the coagulation.

The described method may comprise an additional step: waiting a period of time after the activation of the mixture by means of calcium chloride to allow the compound to coagulate. This may be advisable if a compound with a less liquid consistency is required.

1. Method for the treatment of a joint affected by an articular complaint or an articular pain, which comprises the infiltration in the joint of a compound that comprises at least one blood-derived substance.

2. Method according to claim 1, wherein the blood-derived substance is infiltrated in the joint following the extraction of synovial fluid from said joint.

3. Method according to claim 1, wherein the blood-derived substance is infiltrated in the joint after arthroscopic surgery has been carried out on said joint.

4. Method according to claim 1, wherein the blood-derived substance is autologous.

5. Method according to claim 1, wherein the blood-derived substance is homologous.

6. Method according to claim 1, wherein the blood-derived substance is blood plasma.

7. Method according to claim 6, wherein the blood-derived substance is a platelet-rich plasma (PRP).

8. Method according to claim 7, wherein the blood-derived substance is a plasma rich in growth factors (PRGF).

9. Method according to claim 1, wherein the blood-derived substance is a supernatant of blood plasma.

10. Method according to claim 9, wherein the blood-derived substance is a supernatant of platelet-rich plasma (PRP).

11. Method according to claim 10, wherein the blood-derived substance is a supernatant of plasma rich in growth factors (PRGF).

12. Method according to claim 1, wherein the compound also comprises hyaluronic acid (HA) and/or an HA-derived compound.

13. Method for the treatment of skin, which comprises the external application or the infiltration by means of dermal, subdermal or intramuscular injection of a compound that comprises at least one blood-derived substance.

14. Method according to claim 13, wherein the blood-derived substance is autologous.

15. Method according to claim 13, wherein the blood-derived substance is homologous.

16. Method according to claim 13, wherein the blood-derived substance is blood plasma.

17. Method according to claim 16, wherein the blood-derived substance is a platelet-rich plasma (PRP).

18. Method according to claim 17, wherein the blood-derived substance is a plasma rich in growth factors (PRGF).

19. Method according to claim 13, wherein the blood-derived substance is a supernatant of blood plasma.

20. Method according to claim 19, wherein the blood-derived substance is a supernatant of platelet-rich plasma (PRP).

21. Method according to claim 20, wherein the blood-derived substance is a supernatant of plasma rich in growth factors (PRGF).

22. Method according to claim 13, wherein the compound further comprises hyaluronic acid (HA) and/or at least one HA-derived compound.

23. Compound for the regeneration of tissues, characterised in that it comprises:
at least one blood-derived substance,
hyaluronic acid (HA) and/or at least one HA-derived compound.

24. Compound according to claim 23, wherein the blood-derived substance is autologous.

25. Compound according to claim 23, wherein the blood-derived substance is homologous.

26. Compound according to claim 23, wherein the blood-derived substance is blood plasma.

27. Compound according to claim 26, wherein the blood-derived substance is a platelet-rich plasma (PRP).

28. Compound according to claim 27, wherein the blood-derived substance is a plasma rich in growth factors (PRGF).

29. Compound according to claim 23, wherein the blood-derived substance is a supernatant of blood plasma.

30. Compound according to claim 29, wherein the blood-derived substance is a supernatant of platelet-rich plasma (PRP).

31. Compound according to claim 30, wherein the blood-derived substance is a supernatant of plasma rich in growth factors (PRGF).

32. Method of preparation of a compound for the regeneration of tissues, characterised in that it comprises the following steps:
providing at least one blood-derived substance,
mixing said blood-derived substance with hyaluronic acid (HA) and/or at least one HA-derived compound.

33. Method according to claim 32, wherein the blood-derived substance is autologous.

34. Method according to claim 32, wherein the blood-derived substance is homologous.

35. Method according to claim 32, wherein the HA and/or the HA-derived compound is lyophilized, and the mixing of the blood-derived substance with the HA and/or at least one HA-derived compound is carried out by adding the blood-derived substance to a tube containing the lyophilized HA and/or at least one HA-derived compound and heating said tube to between 25 and 42° C. in order to dissolve the HA and/or at least one HA-derived compound in the blood-derived substance.

36. Method according to claim 32, wherein the HA and/or at least one HA-derived compound is in a solution and the mixing of the blood-derived substance with the HA and/or at least one HA-derived compound is carried out by dissolving the HA and/or at least one HA-derived compound in a sterile saline solution at between 25 and 42° C., and then gently mixing the solution with the PRP.

37. Method according to claim 32, wherein the calcium chloride is applied in a concentration of between 6-120 mM.

38. Method according to claim 32, wherein it comprises the additional step of activating said mixture by means of calcium chloride.

39. Method according to claim 38, wherein it comprises the additional step of waiting for the compound to coagulate, after activating the mixture by means of calcium chloride.
